(19) United States
(12) Patent Application Publication
(10) Pub. No.: US 2022/0304998 A1
(43) Pub. Date: Sep. 29, 2022

(54) REBAMIPIDE FOR USE IN PREVENTION AND TREATMENT OF CROHN'S DISEASE

(71) Applicant: SQUARE POWER LTD., London (GB)

(72) Inventor: Ivan DANEK, Cernosice (CZ)

(21) Appl. No.: 17/638,871

(22) PCT Filed: Sep. 2, 2020
(86) PCT No.: PCT/EP2020/074501

§ 371 Date: Feb. 28, 2022

(30) Foreign Application Priority Data
Sep. 3, 2019 (EP) 19020504.7

(51) Int. Cl. A61K 31/4704; A61P 1/12; A61K 31/606; A61K 31/655; A61K 31/573; A61K 31/519; A61K 38/13; A61K 31/436; A61K 31/4164; A61K 31/496; A61K 39/395

(52) U.S. Cl. CPC A61K 31/4704; A61P 1/12; A61K 31/606; A61K 31/655; A61K 31/573; A61K 31/519; A61K 38/13; A61K 31/436; A61K 31/4164; A61K 31/496; A61K 39/3955

(57) ABSTRACT

The present invention provides rebamipide for use in a method of prevention and/or treatment of Crohn's disease. In particular, rebamipide is used in prevention and/or treatment of Crohn's disease in a person suffering from increased intestinal permeability or in a person who is at risk of increased intestinal permeability.

### FIELD OF THE INVENTION

The present invention relates to rebamipide for use in a method of prevention and/or treatment of Crohn's disease, in particular in a person suffering from increased intestinal permeability or in a person who is at risk of increased intestinal permeability.

### BACKGROUND ART

Crohn's disease (CD) is an illness belonging to the group of inflammatory bowel diseases (IBD). It is a chronic multifactorial disorder, in which genetic, environmental, and microbial factors are involved. While the exact cause is unknown, it seems that the disease onset is triggered by environmental factors that perturb the mucosal barrier, alter the healthy balance of the gut microbiota, and abnormally stimulate gut immune responses. This leads to chronic inflammation of the intestine with typical symptoms such as diarrhea, weight loss, fatigue, rectal bleeding, and abdominal pain.

The disorder may occur at any age although it usually starts in the teens and twenties. It is estimated to affect about 3 in 1,000 people in Europe and North America with a similar frequency in males and females. It most often affects the end of the small intestine and the beginning of the colon, but it may also affect any part of the GI tract from the mouth to the anus. Skip lesions and patchy inflammation are a typical finding in CD.

At present there is no cure for Crohn's disease and current treatment aims at reducing the inflammation that triggers the symptoms. Over the years, several classes of medications have been developed for the treatment of CD. The choice of the medication depends upon the location of inflammation, severity of disease, complications, and the response of the patient to medical treatment. Although mild disease can be treated with 5-aminosalicylates, either alone or in combination with antibiotics, many patients eventually require corticosteroids to control symptoms. They can help reduce inflammation and induce remission but their long-term use is associated with well known adverse effects, and thus are not recommended for maintenance therapy. Moreover, about a half of patients is unable to discontinue corticosteroid therapy without disease exacerbation. Immunosuppressants, such as thiopurines and methotrexate, are frequently prescribed for patients who are resistant to or dependent on corticosteroids; however, these drugs have a slow onset of action, severe adverse effects, and clinical remission rates of about 40%.

Biological therapy using monoclonal antibodies against TNFα, such as infliximab (Remicade) and adalimumab (Humira), is used in patients unresponsive to conventional treatment with corticosteroids and immunosuppressants. Further option are antibodies against interleukins, such as ustekinumab (Stelara) that acts as antagonist of human IL-12 and IL-23, and those binding human integrins, such as vedolizumab (Entyvio) that targets integrin α₄β₇ resulting in gut-selective anti-inflammatory activity. Monoclonal antibodies have relatively fast onset of action and induce mucosal healing, but up to 30% of patients do not respond to the treatment (primary non-responders) and out of those who initially experience a benefit with these drugs up to 50% may lose response to the therapy within twelve months (secondary non-responders), requiring dose escalation or therapy change. Although monotherapy is considered relatively safe, patients are more susceptible to infections and have a higher risk of malignancies. Moreover, treatment withdrawal in patients who have achieved a sustained period of remission remains a persistent problem.

Even though some of the treatments mentioned above may lead to a long-time relief from the symptoms of the disease, most patients with CD ultimately require surgery. Therefore, there is still a high unmet medical need for a treatment that would suppress intestinal inflammation and lead to sustained complete remission of the disease symptoms. To address this need, the present invention provides an affordable treatment that is suitable for both induction and maintenance therapy of CD.

Rebamipide, which is chemically 2-[(4-chlorobenzoyl)amino]-3-(2-oxo-1H-quinolin-4-yl)propanoic acid) is used for the treatment of acute and/or chronic gastritis and gastric ulcers. Its mechanism of action relates to mucosal defense, scavenging free radicals, and temporarily activating genes encoding cyclooxygenase-2.

### SUMMARY OF THE INVENTION

It has been unexpectedly found that rebamipide, when administered to patients suffering from Crohn's disease, is able to suppress the disease symptoms and may even lead to complete remission of the disease. This is very surprising since it hasn't been reported that rebamipide, which is clinically used only for the treatment of gastric conditions, could be effective against Crohn's disease affecting mainly the intestine. Although rebamipide has been marketed since 1990, little is known about its behavior in the GI tract. The molecule is poorly absorbed in the GI tract, which indicates that following oral administration it may reach the intestinal mucosa in a concentration high enough to exert a therapeutic effect. Nevertheless, to the best of my knowledge, peroral rebamipide has never been reported as an effective treatment for Crohn's disease.

The mechanism of action is likely based on rebamipide's ability to induce mucine production in the intestine, to suppress inflammation and to restore the function of the tight junctions of epithelial cells, thus reducing the permeability of the intestinal wall and normalizing bowel function (Diao L et al. Rebamipide suppresses diclofenac-induced intestinal permeability via mitochondrial protection in mice. World J Gastroenterol. 2012; 18(10):1059-1066). This leads to suppression of chronic inflammation and recovery of the intestine, preventing further tissue damage and reduction of the disease symptoms. Indeed, increased small intestinal permeability to various agents is a common observation in Crohn's disease patients (Katz K D et al. Intestinal permeability in patients with Crohn's disease and their healthy relatives. Gastroenterology. 1989; 97(4):927-931) and was reported to precede the onset of the disease in genetically predisposed individuals (Irvine E J & Marshall J K. Increased intestinal permeability precedes the onset of Crohn's disease in a subject with familial risk. Gastroenterology. 2000; 119(6),1740-1744).

The present invention thus provides rebamipide or a pharmaceutical composition thereof for use in a method of prevention and/or treatment of Crohn's disease, in particular by decreasing the intestinal permeability. The method may advantageously be used for induction therapy to reduce inflammation, give relief from symptoms, and/or induce remission as defined below, and/or for maintenance therapy to prevent disease relapse or recurrence in a person who reached remission.

“Rebamipide”, as used herein, shall include all forms of this active ingredient, such as anhydrous form, hydrated or solvated form (e.g. hemihydrate form), crystalline forms; and pharmaceutically acceptable salts thereof.

“Prevention” or “preventive use” shall be understood herein as preventing or delaying the onset of the disease and its symptoms including their recurrence in patients with complete or partial remission after previous treatment. It is also meant to include maintenance of remission and/or non-worsening of the disease symptoms after de-escalation or discontinuation of previous treatment. Furthermore, it includes prevention of disease complications, such as bowel obstructions, ulcers and fistulas, anal fissure, colon cancer, arthritis, uveitis, erythema nodosum, pyoderma gangrenosum, or aphthous stomatitis.

“Treatment” shall be understood herein as a therapy that is able to abrogate, inhibit, slow or reverse the progression of the disease, and/or suppress chronic inflammation in the small intestine or elsewhere in the GI tract. It is also meant to cover amelioration or alleviation of clinical symptoms of the disease, such as abdominal pain, diarrhea, bloating, blood in stool, mouth sores, fatigue, reduced appetite and weight loss, malnutrition, skin inflammation, etc.

The present invention further provides a method for prevention and/or treatment of Crohn's disease by administering a pharmaceutically effective dose of rebamipide or a pharmaceutical composition thereof to a subject in need of such treatment. The subject is preferably a human subject, in particular a person suffering from increased intestinal permeability or a person who is at risk of increased intestinal permeability. Furthermore, the present invention also includes the use of rebamipide in the manufacture of a medicament for preventing and/or treating Crohn's disease, in particular in a person suffering from increased intestinal permeability or in a person who is at risk of increased intestinal permeability.

In one aspect, rebamipide is for use in a method of prevention and/or treatment of Crohn's disease in a person suffering from increased intestinal permeability or in a person who is at risk of increased intestinal permeability, e.g., due to family anamnesis or due to exposure to conditions or substances inducing increased intestinal permeability.

“Increased intestinal permeability” is used herein as a term designating little intestinal wall defects, including those caused by subclinical chronic inflammation (low grade inflammation) of the gut wall. These intestinal wall defects may be manifested by Crohn's disease but also by other medical conditions, such as chronic constipation or gastroparesis. Increased intestinal permeability may be diagnosed using specific tests, such as lactulose-mannitol test (LAMA test; e.g., Sequeira I. R. et al. (2014) PLoS One; 9(6):e99256), A-1-AT test, or zonulin test. Typically, increased intestinal permeability is permeability of the intestinal wall to particles having the size of more than 4 Angstroms in radius.

Substances inducing increased intestinal permeability include non-steroidal anti-inflammatory drugs (NSAIDs), such as acetylsalicylic acid, ibuprofen, naproxen, ketoprofen, fenoprofen, flurbiprofen, diclofenac, ketorolac, etodolac, indomethacin, tolmetin, piroxicam, meloxicam and selective COX-2 inhibitors such as celecoxib and etoricoxib; alcohol; nicotine; food additives; antibiotics; and chemotherapeutics. Thus, simultaneous or sequential co-administration of rebamipide with non-steroidal anti-inflammatory drugs, chemotherapeutics or antibiotics prevents intestinal wall damage and thus prevents or delays the onset of Crohn's disease associated with increased intestinal permeability. Prophylactic use of rebamipide may also be useful in persons abusing alcohol, nicotine or other drugs known to damage intestinal wall. The term “abuse” as used herein is meant to include any consumption, which is not necessary for medical reasons and leads to dependency and/or health impairments including low grade inflammation of the gut wall.

Conditions inducing increased intestinal permeability are related to stress, imbalanced diet, allergy, bacterial, viral or parasitic infections and various medical treatments. Such conditions in particular include stress-induced gastritis, alimentary intoxication, disbalance of cholic acids, gastric HCl and pepsin secretion, non-infectious diarrhea, radiation therapy, chemotherapy, infectious or post-infectious impairment of the GIT mucosa, dysmicrobia (e.g. induced by antibiotic treatment).

The person suffering from increased intestinal permeability typically suffers from at least one condition selected from low grade inflammation of the gut wall, chronic constipation or gastroparesis.

In one aspect, rebamipide is for use in a combination therapy with at least one other drug effective against Crohn's disease, wherein said other drug can be administered simultaneously, separately or sequentially to rebamipide. The combination therapy is preferably used in a method of treatment of active Crohn's disease. More preferably, the combination therapy is used for induction therapy to reduce inflammation, give relief from symptoms, and/or induce remission.

The term drug is meant to encompass both small molecules, i.e. having molecular weight less than about 900 g/mol, and macromolecules, especially antibodies. The at least one other drug effective against Crohn's disease is preferably selected from 5-aminosalicylates, corticosteroids, immunosuppressants, antibiotics, and antibodies. More preferably, said other drug is selected from corticosteroids, immunosuppressants, and antibodies. Most preferably, said other drug is a corticosteroid.

5-aminosalicylates are anti-inflammatory chemotherapeutic agents that are mainly used in the management of mild to moderate Crohn's disease. These compounds include mesalazine (i.e. 5-aminosalicylic acid, 5-ASA, or mesalamine) and conjugated derivatives thereof, known for their anti-inflammatory properties, such as sulfasalazine, olsalazine, and balsalazide. Preferably, the 5-aminosalicylate is selected from mesalazine and sulfasalazine.

Corticosteroids are a class of anti-inflammatory medications that are used to help reduce inflammation in the digestive tract and relieve symptoms. The corticosteroid to be used in a combination with rebamipide according to the present invention is preferably selected from prednisone, prednisolone, methylprednisolone, hydrocortisone, or budesonide. It can be advantageously further combined with at least one thiopurine to provide a triple combination. Corticosteroids may be administered orally, rectally, or intravenously.

In one embodiment, the corticosteroid is in an oral dosage form (e.g. in a tablet or capsule). This form is typically administered to a person with moderate to severe active disease. Oral budesonid is also advantageously used in mild or moderate disease that affects the end of the small intestine and the beginning of the colon.

In another embodiment, the corticosteroid is in a rectal form (e.g. in a suppository, enema, or rectal foam), and is preferably selected from hydrocortisone, methylprednisolone, and budesonide. Rectal forms are especially suitable for people who have active disease that affects the lower part of the colon or the rectum. It specifically targets the area affected by the disease, and is thus associated with fewer side effects than the oral forms.

In another embodiment, the corticosteroid is administered intravenously and is preferably selected from hydrocortisone and methylprednisolone. The intravenous administration is especially suitable for patients with severe active disease.

Immunosuppressants are drugs that inhibit the activity of the immune system, thus reducing inflammation. They are preferably selected from thiopurines, methotrexate, cyclosporine, and tacrolimus. Thiopurines and methotrexate are particularly preferred. Thiopurines are purine antimetabolites, preferably selected from azathioprine and 6-mercaptopurine. They can be advantageously combined with rebamipide and at least one corticosteroid or at least one antibody to provide a triple combination.

Antibiotics are primarily used in mild to moderate disease as they help to lower the amount and change the composition of bacteria in the intestines, resulting in relief of symptoms. They are also used to control infections and Crohn's disease complications, such as abscesses, fistulas, and pouchitis. Preferably, the antibiotic is selected from metronidazole, ciprofloxacin, rifaximin, and ampicillin. Metronidazole and ciprofloxacin are particularly preferred, especially in intravenous dosage form.

Antibodies (biologics) are protein molecules targeting specific mediators of the disease, such as cytokines or integrins. They can be of any origin, such as from human or mouse, including humanized and chimeric antibodies. The term “antibody”, as used herein, is meant to include also antigen-binding fragments of the antibodies, i.e. fragments comprising the variable region of the antibody, such as F(ab′)2, Fab, Fab′, Fv and scFv fragments, and derivatives, such as those that have been chemically altered, e.g. pegylated.

In one embodiment, the antibody is selected from antibodies against TNFα, preferably from infliximab, adalimumab, golimumab, certolizumab pegol and biosimilars thereof. More preferably, the antibody is selected from infliximab, adalimumab, and biosimilars thereof.

In another embodiment, the antibody is selected from antibodies against interleukins, such as IL-12 and/or IL-23. Preferably, the antibody is ustekinumab or a biosimilar thereof.

In yet another embodiment, the antibody is selected from antibodies against integrins, especially against integrin α₄β₇. Preferably, the antibody is selected from natalizumab, vedolizumab, and biosimilars thereof. More preferably, the antibody is vedolizumab or a biosimilar thereof.

More particularly, the combination therapy with at least one other drug effective against Cohn's disease as defined above comprises the steps of administering rebamipide to a person being treated with a dose of at least one other drug effective against Crohn's disease; and decreasing the dose of said other drug.

Advantageously, the combination therapy comprises the steps of administering rebamipide as an add-on to a treatment with at least one other drug effective against Crohn's disease, especially with 20 to 60 mg of prednisone or equivalent dose of another corticosteroid; and decreasing the dose of said other drug.

In one embodiment, the dose of said other drug is decreased at the beginning of rebamipide administration, i.e. at same time the person starts being given rebamipide.

In another embodiment, the dose of said other drug is decreased after symptom improvement, as can be defined by a decrease in CDAI by more than 100 points or by a decrease in fecal calprotectin by more than 150 μg/g.

The at least one other drug may be completely discontinued after the patient reaches remission, as defined by endoscopy (mucosal healing) and/or CDAI lower than 150 points (clinical remission) and/or fecal calprotectin lower than 200 μg/g feces (inflammation suppression).

The combination therapy may also be advantageously used for dose-sparing of the other drug, especially for dose-sparing of a corticosteroid to prevent its adverse effects. Thus, the present invention further provides rebamipide for use in a method of dose-sparing of at least one other drug effective against Crohn's disease. The method advantageously comprises the steps of administering rebamipide to a person being treated with a dose of said other drug effective against Crohn's disease, preferably with 20 to 60 mg of prednisone or equivalent dose of another corticosteroid; and decreasing the dose of said other drug.

In one aspect, rebamipide is for use in a monotherapy, i.e. the subject to be treated is administered only rebamipide and does not receive any other drug effective against Crohn's disease. Rebamipide monotherapy is preferably used in a method of prevention of Crohn's disease, including prevention of the first disease outbreak in a person susceptible to the disease, e.g. due to having or being at risk of increased intestinal permeability, and/or prevention of the disease relapse or recurrence in a person who responded to induction treatment and reached remission as defined above. More preferably, rebamipide monotherapy is used for maintenance therapy of Crohn's disease to prevent inflammation and/or maintain remission.

The present invention further provides a therapeutic combination comprising rebamipide and at least one other drug effective against Crohn's disease. The term drug is meant to encompass both small molecules, i.e. having molecular weight less than about 900 g/mol, and macromolecules, such as antibodies, preferably selected from antibodies against TNFα, interleukins, or integrins, such as integrin α₄β₇. The at least one other drug is selected from 5-aminosalicylates, preferably sulfasalazine or mesalazine; corticosteroids, preferably prednisone, prednisolone, methylprednisolone, hydrocortisone, or budesonide; immunosuppressants, preferably azathioprine, 6-mercaptopurine, methotrexate, cyclosporine and tacrolimus; antibiotics, preferably metronidazole and ciprofloxacin; and antibodies (biologics), preferably infliximab, adalimumab, vedolizumab, and biosimilars thereof.

In a preferred embodiment, the combination comprises rebamipide and at least one other drug selected from corticosteroids, thiopurines, and antibodies. In a more preferred embodiment, the present invention provides a therapeutic combination comprising rebamipide and at least one corticosteroid, especially 20 to 60 mg of prednisone or equivalent dose of another corticosteroid.

In the therapeutic indications as described in the present invention, rebamipide may preferably be used in pharmaceutical forms for oral administration such as tablets, capsules, dragees, granules, microgranules (sachets), orodispersible tablets or films, sublingual tablets, crushed tablets, oral solutions, oral suspensions, syrups, mouthwashes or rinses. Preferably, the oral pharmaceutical forms, such as tablets, capsules, dragees and granules, is a form with enteric release, such as enteric sustained release or enteric controlled release.

The pharmaceutical forms may contain at least one pharmaceutically acceptable excipient selected from fillers, binders, lubricants, glidants, disintegrants/swelling agents, solubilizers, enteric release agents, mucoadhesive components, sustained release agents, preservatives, coatings and colorants. Such excipients are known in the art of pharmaceutical formulation, and the skilled person is capable of selecting suitable excipients for the relevant pharmaceutical forms.

Suitable methods for preparing the pharmaceutical forms and compositions includes the processes of wet granulation or dry granulation of the active ingredient with the auxiliary substances and components, or direct homogenization of the active ingredient with the auxiliary substances and components.

Fillers may preferably be selected from saccharide alcohols (such as mannitol, sorbitol, xylitol), lactose, starch, pregelatinized starch, cellulose, silicified cellulose, calcium hydrogen phosphate, calcium phosphate, sucrose and calcium sulphate. The fillers may preferably be present in the amount of 5 to 90 wt. %, relative to the total weight of the composition.

Binders may preferably be selected from starch, pregelatinized starch, povidone, copovidone, hydroxypropyl methylcellulose, hydroxypropyl cellulose, ethyl cellulose, cellulose. The binders may preferably be present in the amount of 1 to 20 wt. %, relative to the total weight of the composition.

Lubricants may preferably be selected from magnesium stearate, calcium stearate, stearic acid, polyethylene glycol and sodium stearyl fumarate. The lubricants may preferably be present in the amount up to 5 wt. %, relative to the total weight of the composition.

Glidants may preferably be selected from silica, talc and sodium lauryl sulphate. The glidants may preferably be present in the amount of 0.5 to 10 wt. %, relative to the total weight of the composition.

Swelling and/or disintegrating agents may preferably be selected from crospovidone, copovidone, povidone, croscarmellose, hydroxypropyl methylcellulose, starch, pregelatinized starch, low-substituted hydroxypropyl cellulose, sodium carboxymethyl starch. The swelling/disintegrating agents may preferably be present in the amount of 1 to 50 wt. %, relative to the total weight of the composition.

Solubilizers may preferably be selected from poloxamer, sodium lauryl sulphate, polysorbate, polyoxylated oleic glycerides, glycerol monostearate and cyclodextrins. The solubilizers may preferably be present in the amount up to 30 wt. %, relative to the total weight of the composition.

Enteric release agents may preferably be selected from hydroxypropyl methylcellulose phthalate, poly(methacrylic acid-co-methyl methacrylate), cellulose acetate phthalate, poly(vinyl acetate phthalate), esters of aleuritic acid. The enteric release agents may preferably be present in the amount of 2 to 40 wt. %, relative to the total weight of the composition.

Mucoadhesive components may preferably be selected from propylene glycol alginate, sodium alginate, calcium alginate, potassium alginate, hydroxypropyl methylcellulose, sodium carmellose, polyacrylic acid, polyethylene oxide, povidone and copovidone. The mucoadhesive components may preferably be present in the amount of 5 to 70 wt. %, relative to the total weight of the composition.

Sustained release agents may preferably be selected from cellulose and cellulose ethers such as hydroxypropyl cellulose, hydroxypropyl methylcellulose, carboxymethylcellulose, sodium carboxymethylcellulose, ethyl cellulose, methylcellulose, polyvinyl acetate, alginic acid, propylene glycol alginate, sodium alginate, calcium alginate, potassium alginate, polymethacrylates, guar gum, xanthan gum, carrageenan, castor oil, beeswax, carnauba wax, glycerol palmitostearate, glycerol monostearate, glycerol behenate, stearyl alcohol, polyacrylic acid. The sustained release agents may preferably be present in the amount of 5 to 70 wt. %, relative to the total weight of the composition.

The oral pharmaceutical composition may in some embodiments further contain a pharmaceutically acceptable component capable of forming carbon dioxide upon contact with gastric juices, such component may preferably be selected from carbonates and hydrogen carbonates of alkali metals and alkaline earth metals; and may preferably be present in an amount in the range from 1 to 50 wt. %, relative to the total weight of the composition.

A typical daily dose of rebamipide may range from 1 to 5000 mg for an average human (70 kg weight), more preferably from 50 to 2500 mg, even more preferably from 100 to 1000 mg, and most preferably from 300 to 600 mg or from 300 to 900 mg.

When immediate release formulation of rebamipide is administered, the daily dose is typically divided into several doses, which are administered separately. The daily dose may be divided into two to six separate doses taken twice daily or three times per day or four times per day or five times per day or six times per day. In a preferred embodiment the daily dose is divided into three separate doses administered three times per day, e.g. 100 mg dose administered three times per day. Alternatively, the whole daily dose can be taken at once, especially if it is in the form of a sustained release formulation. e.g. 300 mg dose administered once daily.

Hereinafter, the present invention will be described more specifically by the following working examples. However, the following working examples are provided only for illustrations and thus the present invention is not limited to it.

### EXAMPLES

Several patients suffering from Crohn's disease for at least 5 years without sufficient response to standard treatment were given rebamipide tablets three times a day. These patients had long history of treatment with various medicaments but were not able to reach remission after relapse of the disease using standard options. The severity of the disease was quantified by the CDAI, and the level of inflammation by fecal calprotectin.

Crohn's Disease Activity Index (CDAI) is a research tool used to quantify the symptoms of patients with Crohn's disease. It consists of eight factors, each summed after adjustment with a weighting factor. Remission of Crohn's disease is defined as CDAI below 150, whereas severe disease is defined as a value of greater than 450.

Calprotectin (FC) is a small calcium-binding protein that is used as a biomarker for inflammatory bowel diseases since its fecal concentration correlates well with the disease severity. Calprotectin values lower than 200 μg/g feces are considered the normal level. It was measured in stool samples using commercially available enzyme-linked immunosorbent assay (ELISA) kit (EK-CAL, Bühlmann).

### Example 1

A female patient, age 39, suffering from Crohn's ileocolitis, was diagnosed 16 years ago. Over the course of the disease she was given various medications including peroral, parenteral and topical corticosteroids, azathioprine, and TNFα inhibitors infliximab and adalimumab. After immunological response had developed against these antibodies, affecting skin and joints, the patient switched to vedolizumab (Entyvio), that was able to stabilize her for the next 22 months. During the relapse of the disease, the patient suffered with abdominal pain, increased stool frequency, joint and muscle pain, subfebrile fever, fatigue, and dry dermatitis with fecal calprotectin levels up to 1939 μg/g and CDAI of 358 points. Her treatment continued with methylprednisolon 24 mg/day without success. Concomitantly with the corticosteroid treatment she started being given rebamipide 100 mg three times daily resulting in fast remission of the disease as evidenced by alleviation of the disease symptoms (CDAI of 181 points) and decrease of calprotectin level to 228 μg/g after 6 weeks of the treatment. The corticosteroid dose was then reduced to half without any adverse effect. Corticosteroids were completely withdrawn after another 6 weeks when the calprotectin level lowered to 63 μg/g and CDAI to 73 points and the patient continued only rebamipide. No worsening in disease activity has been observed so far.

### Example 2

A male patient, age 27, was diagnosed with Crohn's ileocolitis 5 years ago and treated with mesalazine (Pentasa), budesonid, and metronidazole. He developed pancreatitis after three months on mesalazine. The medication was subsequently switched to azathioprine combined with prednisone and later to methotrexate. However, the medication was not well tolerated and the patient was indicated for biological therapy. He was treated with infliximab (Remicade) and later with adalimumab (Humira). After the last relapse (calprotectin 2215 μg/g; CDAI of 479 points) he was given prednisone 40 mg/day but was not able to reach remission. He started taking rebamipide 200 mg t.i.d. as an add-on to prednisone. After 6 weeks (calprotectin 356 μg/g; CDAI of 225 points) the rebamipide dose was adjusted to 100 mg t.i.d. and prednisone dose to 30 mg/day. Prednisone dose was then lowered by 10 mg every 2 weeks and completely discontinued after another 6 weeks (calprotectin 110 μg/g; CDAI of 84 points). The patient continued only rebamipide 100 mg t.i.d. No worsening in disease activity has been observed so far.

### Example 3

A female patient, age 32, was diagnosed with Crohn's ileitis 12 years ago. Therapy with budesonide and mesalazine was able to maintain remission for about 10 years. She was thereafter hospitalized with abdominal pain and loose stool 3-4 times a day, with calprotectin level up to 1051 μg/g and CDAI of 377 points. She was prescribed azathioprine but it had to be discontinued after two weeks due to acute pancreatitis. Her treatment continued with prednisone 50 mg/day concomitantly with rebamipide 100 mg three times daily. The prednisone dose was adjusted to 25 mg after 14 days due to alleviation of the clinical symptoms and completely withdrawn after another 6 weeks with calprotectin level lowered to 122 μg/g and CDAI of 98 points and she continued only rebamipide. No worsening in disease activity has been observed so far.

The results obtained with the patient sample show that rebamipide is able to manage the disease symptoms, normalize calprotectin levels and induce remission even in those patients, who exhausted standard treatment options. The treatment started as an add-on to corticosteroid treatment due to ethical reasons but it is believed that it would be efficient even if prescribed as the only treatment. It is important to note that the corticosteroid treatment alone was not able to completely suppress the disease symptoms and neither patient had calprotectin lower than 200 μg/g feces, and CDAI lower than 150 points using only corticosteroids. Moreover, long-term therapy with rebamipide alone seems to be well tolerated and able to maintain remission.

1. A method of prevention and/or treatment of Crohn's disease comprising administration of rebamipide.

2. The method according to claim 1, wherein rebamipide is combined with at least one other drug effective against Crohn's disease.

3. The method according to claim 2, wherein it comprises the steps of administering rebamipide to a person being treated with a dose of at least one other drug effective against Crohn's disease; and decreasing the dose of said other drug.

4. The method according to claim 3, wherein the dose of said other drug is decreased at the beginning of rebamipide administration.

5. The method according to claim 3, wherein the dose of said other drug is decreased after symptom improvement.

6. The method according to claim 2, wherein said other drug is discontinued after the person reaches remission.

7. The method according to claim 2, wherein rebamipide is used for treatment of Crohn's disease.

8. The method according to claim 7, wherein rebamipide is adminstered to a person with active Crohn's disease to induce remission.

9. The method according to claim 1, wherein the method is a monotherapy.

10. The method according to claim 9, wherein rebamipide is used for prevention of Crohn's disease.

11. The method according to claim 9, wherein rebamipide is adminstered to a person in remission of the disease to prevent disease relapse and/or recurrence.

12. (canceled)

13. The method according to claim 2, wherein said other drug is selected from 5-aminosalicylates, preferably selected from sulfasalazine and mesalazine; corticosteroids, preferably selected from prednisone, prednisolone, methylprednisolone, hydrocortisone and budesonide; immunosuppressants, preferably selected from thiopurines, methotrexate, cyclosporine and tacrolimus; antibiotics, preferably selected from metronidazole and ciprofloxacin; and antibodies, preferably selected from infliximab, adalimumab, vedolizumab and biosimilars thereof.

14. The method according to claim 13, wherein said other drug is selected from corticosteroids, immunosuppressants, and antibodies.

15. The method according to claim 13, wherein said other drug is a corticosteroid, preferably 20 to 60 mg of prednisone or equivalent dose of another corticosteroid.

16. The method according to claim 1, wherein rebamipide is used in prevention and/or treatment of Crohn's disease in a person suffering from increased intestinal permeability or in a person who is at risk of increased intestinal permeability.

17. The method according to claim 16, wherein the person suffering from increased intestinal permeability is a person suffering from at least one condition selected from low grade inflammation of the gut wall, chronic constipation or gastroparesis.

18. The method according to claim 16, wherein the person at risk of increased intestinal permeability is a person suffering from stress, imbalanced diet, bacterial, viral or parasitic infection.

19. The method according to claim 16, wherein the person at risk of increased intestinal permeability is a person exposed to at least one substance selected from non-steroidal anti-inflammatory drugs, alcohol, nicotine, food additives, chemotherapeutics and antibiotics.

20. The method according to claim 16, wherein the person at risk of increased intestinal permeability is a person suffering from or exposed to at least one condition selected from stress-induced gastritis, alimentary intoxication, disbalance of cholic acids, gastric HCl and pepsin secretion, non-infectious diarrhea, radiation therapy, chemotherapy, infectious or post-infectious impairment of the GIT mucosa, dysmicrobia.

21. The method according to claim 1, wherein rebamipide is administered in an oral pharmaceutical form, preferably selected from tablets, capsules, dragees, granules, microgranules (sachets), orodispersible tablets or films, sublingual tablets, crushed tablets, oral solutions, oral suspensions, syrups, mouthwashes or rinses.

22. (canceled)

23. (canceled)

24. (canceled)

25. (canceled)

26. The method according to claim 1, wherein rebamipide is administered in a daily dose of 1 to 5000 mg, more preferably from 50 to 2500 mg, even more preferably from 100 to 1000 mg, and most preferably from 300 to 600 mg.
